(19) United States
(12) Patent Application Publication
(10) Pub. No.: US 2009/0028993 A1
(43) Pub. Date: Jan. 29, 2009

(54) Lactobacillus buchneri strain LN1326 and its use to improve aerobic stability of silage

(75) Inventors: Russell K. Chan, San Diego, CA (US); Scott M. Dennis, Urbandale, IA (US); Elizabeth K. Harman, New Virginia, IA (US); Carol A. Hendrick, Des Moines, IA (US); Barbara G. Ruser, Buxtehude (DE); William Rutherford, Des Moines, IA (US); Brenda K. Smiley, Granger, IA (US); Cora Wortman, Brooklyn Park, MN (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International, Inc.

(21) Appl. No.: 11/949,803

(22) Filed: Dec. 4, 2007

Related U.S. Application Data
(60) Provisional application No. 60/869,376, filed on Dec. 11, 2006.

(51) Int. Cl. A23K 1/16; A23K 1/14; A23K 3/02; C12N 1/20

(52) U.S. Cl. 426/2; 426/61; 435/252.5; 426/53

(57) ABSTRACT

A method for treating silage to enhance aerobic stability by inhibiting growth of microorganisms selected from yeasts, molds and spore-forming bacteria is disclosed. The method comprises treating silage or feed with a composition comprising Lactobacillus buchneri, LN 1326, or the antimicrobial components produced thereby. The strain of Lactobacillus buchneri disclosed in the invention has been purified and isolated and has been found to be nontoxic, safe and able to improve aerobic stability of silage.

### CROSS-REFERENCE TO RELATED APPLICATION

This application claims the benefit of U.S. Provisional Application No. 60/869,376, filed Dec. 11, 2006 which is herein incorporated by reference in its entirety.

### TECHNICAL FIELD

This invention relates generally to the silage process and to microorganisms and use of the same in treating animal feed and silage to enhance aerobic stability of the same.

### BACKGROUND OF INVENTION

The ensiling process is a method of moist forage preservation and is used all over the world. Silage accounts for more than 200 million tons of dry matter stored annually in Western Europe and the United States alone. The concept involves natural fermentation, where lactic acid bacteria ferment water soluble carbohydrates to form organic acids under anaerobic conditions. This causes a decrease in pH which then inhibits detrimental microbes so that the moist forage is preserved. The process can be characterized by four different phases.

Upon sealing in the storage unit, the first phase is aerobic, when oxygen is still present between plant particles and the pH is 6.0 to 6.5. These conditions allow for continued plant respiration, protease activity and activity of aerobic and facultative aerobic microorganisms. The second phase is fermentation, which lasts several days to several weeks after the silage becomes anaerobic. Lactic acid bacteria develop and become the primary microbial population thereby producing lactic and other organic acids, decreasing the pH to 3.8 to 5.0. The third phase is stable with few changes occurring in the characteristics of the forage so long as air is prevented from entering the storage unit. The final phase is feedout, when the silage is ultimately unloaded and exposed to air. This results in reactivation of aerobic microorganisms, primarily yeast, molds, bacilli and acetic acid bacteria which can cause spoilage.

Aerobic instability is the primary problem in silage production. Even before storage units are open for feedout, silage can be exposed to oxygen because of management problems (i.e., poor packing or sealing). Under these types of aerobic conditions, rapid growth of yeast and mold cause silages to heat and spoil, decreasing its nutritional value. Aerobic instability can be a problem even in inoculated silage that has undergone what would traditionally be considered a “good” fermentation phase, namely a rapid pH drop, and a low terminal pH. The yeast which contribute to instability in these conditions may be those which are tolerant of acid conditions and can metabolize the lactic acid produced by lactic acid bacteria during fermentation.

Management techniques that can be used to help prevent this condition involve using care to pack the silage well during the ensiling process and, also, using care in removing silage for feeding to minimize the aeration of the remaining silage.

It is possible to use both chemical and biological additives in making silage to promote adequate fermentation patterns especially under sub-optimal conditions. Biological additives comprise bacterial inoculants and enzymes. Bacterial inoculants have advantages over chemical additives because they are safe, easy to use, non-corrosive to farm machinery, they do not pollute the environment and are regarded as natural products. Silage inoculants containing principally homofermentative lactic acid bacteria have become the dominant additives in many parts of the world. Their function is to promote rapid and efficient utilization of a crop's water soluble carbohydrates resulting in intensive production of lactic acid and a rapid decrease in pH. Inoculants also reduce aerobic spoilage and improve animal performance.

The concept of heterofermentative lactic acid bacteria in an inoculant has gained recent favor. The idea is that increased levels of undissociated volatile fatty acids, such as acetate, may inhibit other microbes that initiate aerobic deterioration. Heterofermenters have the ability to convert lactic acid to acetic acid in the presence of oxygen, and the acetate produced may inhibit other deleterious organisms. With such a mechanism, one-third of the lactic acid dry matter consumed will be lost as carbon dioxide. However a small loss of 1% or perhaps up to 2% dry matter may easily offset much larger losses by aerobic microorganisms. Concerns with heterofermentative lactic acid bacteria include effects on animal performance as well as the identification of appropriate strains useful for the procedure. Different strains of even the same species do not have identical properties and vary in their fermentation characteristics.

A review of the silage process and the use of inoculants can be found in Weinberg, ZNG. & Muck, RE. (1996) FMS Microbiology Rev. 19:53-68, the disclosure of which is incorporated herein by reference.

The ensiling process is a complex one and involves interactions of numerous different chemical and microbiological processes. Further, different silages and different methods of ensiling present a variety of different needs. A need exists in the art for further improvement in compositions and methods to improve the aerobic stability of silage.

### SUMMARY OF THE INVENTION

Embodiments of the invention include compositions for use as silage inoculants comprising silage quality preserving amounts of Lactobacillus buchneri strain LN1326 (hereafter LN1326), having Patent Deposit No. NRRL B-30989, or a mutant thereof which retains the silage preservative activity of LN1326, and carrier. Such compositions may contain about 10² to about 10¹² viable organisms per gram wet weight of silage optionally about 10⁷ to about 10¹⁰ viable organisms per gram wet weight of silage, for example about 10⁹ to about 10¹⁰ viable organisms per gram wet weight of silage. The carrier in the compositions of the embodiments may be a liquid or a solid, such as, but not limited to, calcium carbonate, starch, and cellulose.

Another embodiment of the invention is a biologically pure culture of LN1326, having Patent Deposit No. NRRL B-30989.

Embodiments of the invention include methods for treating silage by inhibiting the growth thereon of spoilage organisms selected from yeasts, molds and spore-forming bacteria, which comprises: adding to the silage a spoilage organism inhibiting amount of the compositions of the embodiments. The silage to be treated by the methods of the embodiments may be made from a variety of plant sources, including but not limited to, grass, maize, alfalfa, wheat, legumes, sorghum, sunflower and barley. The compositions of the embodiments may also be added to the silage upon storage. The silage may be ensiled in a variety of ways, including in the form of a bale, a bag, a bunker, a stave silo, or a silo. The methods of treating silage using the compositions of the embodiments include adding to the silage a silage quality preserving amount of LN1326.

Embodiments of the invention further include silage comprising a silage quality preserving amount of LN1326 or a silage quality preserving amount of a mutant thereof. The silage included in the embodiments may be a component of animal feed.

Embodiments of the invention also include compositions for use as silage inoculants comprising LN1326 combined with a ferulate esterase producing bacterial strain or a functional mutant thereof and a suitable carrier. The ferulate esterase strain may be, for example, a Lactobacillus strain or a functional mutant thereof, such as a Lactobacillus strain selected from the group consisting of L. buchneri, L. plantarum, L. brevis, L. reuteri, L. alimentarius, L. crispatus, and L. paralimentarius. Such strains may include, for example, those selected from the group consisting of L. buchneri, strain LN4017 (Patent Deposit No. PTA-6138), L. plantarum, strain LP678 (Patent Deposit No. PTA-6134), L. plantarum, strain LP3710 (Patent Deposit No. PTA-6136), L. plantarum, strain LP3779 (Patent Deposit No. PTA-6137), L. plantarum, strain LP7109 (Patent Deposit No. PTA-6139), L. brevis, strain LB1154 (Patent Deposit No. NRRL B-30865), L. buchneri, strain LN4888 (Patent Deposit No. NRRL B-30866), L. reuteri, strain LR4933 (Patent Deposit No. NRRL B-30867), L. crispatus L12127 (Patent Deposit No. NRRL B-30868), L. crispatus, strain L12350 (Patent Deposit No. NRRL B-30869), L. crispatus, strain L12366 (Patent Deposit No. NRRL B-30870), Lactobacillus species unknown, strain UL3050 (Patent Deposit No. NRRL B-30871), and mixtures thereof. Such compositions may include about 10¹ to about 10¹⁰ viable organisms of the bacterial strains or functional mutants thereof per gram of a pre-ensiled plant material. Optionally, they may include from about 10² to about 10⁷ viable organisms of the bacterial strains or functional mutants thereof, for example from about 10³ to about 10⁶ viable organisms of the bacterial strains or functional mutants thereof per gram of a pre-ensiled plant material.

Additionally, another embodiment is a silage inoculant, comprising viable cultures of a homofermentive lactic acid bacteria and a heterofermentive lactic acid bacteria, wherein the homofermentive lactic acid bacteria are isolated and pure L. plantarum (such as, for example, LP286 (ATCC Patent Deposit No. 53187), LP287 (ATCC Patent Deposit No. 55058), LP329 (ATCC Patent Deposit No. 55942), LP346 (ATCC Patent Deposit No. 55943), LP347 (ATCC Patent Deposit No. 55944), or a functional mutant thereof) and the heterofermentive lactic acid bacteria are isolated and pure L. buchneri strain LN1326, and wherein the ratio of viable cells of the homofermentive lactic acid bacteria to the heterofermentive lactic acid bacteria ranges from about 1:5 to about 1:15, about 1:8 to about 1:12, or about 1:10. The silage inoculant of this embodiment can optionally comprise a viable culture of Enterococcus faecium, such as, for example, EF301 (ATCC Patent Deposit No. (55593), EF202 (ATCC Patent Deposit No. 53519), or a functional mutant thereof. The silage inoculant may also comprise a carrier. An additional embodiment discloses such a silage inoculant with at least two strains of homofermentive lactic acid bacteria, such as, for example, at least two of LP286 (ATCC Patent Deposit No. 53187), LP287 (ATCC Patent Deposit No. 55058), LP346 (ATCC Patent Deposit No. 55943), LP347 (ATCC Patent Deposit No. 55944), LP329 (ATCC Patent Deposit No. 55942), or a functional mutant thereof. Further, this inoculant may optionally comprise at least one strain of Enterococcus faecium, such as, for example, EF301, EF202, and functional mutants thereof. Additional embodiments include animal feed or silage comprising this silage inoculant.

An exemplary embodiment is an animal feed or silage comprising an isolated and purified combination of a viable culture of at least two of (a) LP286 (ATCC Patent Deposit No. 53187), LP287 (ATCC Patent Deposit NQ. 55058), LP346 (ATCC Patent Deposit No. 55943), LP347 (ATCC Patent Deposit No. 55944), LP329 (ATCC Patent Deposit No. 55942), or a functional mutant thereof; and (b) L. buchneri strain LN1326 or a functional mutant thereof, wherein the ratio of viable cells of (a) to (b) ranges from about 1:5 to about 1:15. The animal feed or silage of these embodiments may be, for example, whole plant corn silage or high moisture corn.

A method of the embodiments is a method of treating animal feed or silage, comprising administering the silage inoculant containing LN1326 to the feed or silage at about 1×10⁴ to 1×10⁵ CFU/g of feed or silage. Additionally, another method of the embodiments is a method of improving animal performance, comprising feeding the animal the animal feed that has been inoculated with the silage inoculants as described in the other embodiments.

### DETAILED DESCRIPTION OF THE INVENTION

A microorganism has been isolated and purified which improves the aerobic stability of ensiled forage. A specific strain of the species L. buchneri has been shown to enhance aerobic stability of silage by not only metabolizing lactic acid but also by producing a substance which is toxic to microorganisms that contribute to causing aerobic instability in silage. While not wishing to be bound by any one theory, it is likely that a combination of metabolites (predominantly volatile fatty acids) are responsible for this effect. Furthermore, the metabolism of L. buchneri is believed to produce both acetic acid and propionic acid, both of which are known to inhibit the growth of yeast and molds.

In embodiments of the present invention, the inhibition of organisms responsible for spoilage is accomplished by treating the silage with organisms of the species L. buchneri, especially the strain LN1326 or with compositions containing LN1326 or closely related organisms, and as well by treatment with effective mutants or equivalents of LN1326 and compositions containing same.

The compositions which are used in the embodiments of the invention may be in either liquid or dry form and may contain additional bacterial strains. In solid treatment forms, the composition may comprise LN1326 together with a carrier. The carrier may be in the nature of an aqueous or nonaqueous liquid or a solid. In solid forms, the composition may contain solid carriers or physical extenders. Examples of such solid carriers, solid diluents or physical extenders include maltodextrin, starches, calcium carbonate, cellulose, whey, ground corn cobs, and silicone dioxide. In short, the carrier may be organic or an inorganic physical extender. The solid composition can be applied directly to the forage in the form of a light powder dusting, or if it is disbursed in a liquid carrier, it can successfully be sprayed on the forage.

Typical compositions useful for treating silage according to the embodiments contain about 10² to about 10¹² viable organisms/g, including about 10⁷ to about 10¹⁰ viable organisms/g, and also about 10⁹ to about 10¹⁰ viable organisms/g in soluble formulations. For granular formulations, the range of about 10⁴ to about 10¹⁰ viable organisms is encompassed, as well as about 10⁷ to about 10⁸.

The treatment range for silage is typically about 10⁷ to about 10¹⁷ viable organisms/ton, such as about 10⁹ to about 10¹⁵ viable organisms/ton, and also including about 10¹⁰ to about 10¹² viable organisms/ton.

Those of ordinary skill in the art will know of other suitable carriers and dosage forms, or will be able to ascertain such, using routine experimentation. Further, the administration of the various compositions can be carried out using standard techniques common to those of ordinary skill in the art.

As used herein the term “strain” shall be interpreted to include any mutant or derivative of the various bacterial strains disclosed herein, for example, L. buchneri strain LN1326 (Patent Deposit No. NRRL B-30989), which retains the functional activity of improving aerobic stability of forage as described and defined by the methods and examples disclosed herein.

The LN1326 microorganism of the embodiments was purified and isolated from grass. After much experimentation it was discovered from testing a collection of isolates.

After purification and isolation of the specific strain, taxonomic studies were done to identify the strain. It was identified as L. buchneri and given the prototype number LN1326. According to the invention, this strain, compositions comprising this strain, or the factors produced by this strain, are used to treat forage materials.

Materials that are suitable for ensiling or storage, according to the methods of the invention, are any which are susceptible to aerobic spoilage. The material will usually contain at least 25% by weight dry matter. Such materials include rye or traditional grass, maize, including high moisture corn, whole plant corn, alfalfa, wheat, legumes, sorghum, sunflower, barley or other whole crop cereals. The silage may be in bales (a form particularly susceptible to aerobic spoilage), oxygen limiting bags, bunkers, upright stave silos, oxygen limiting silos, bags, piles or any other form of storage which may be susceptible to aerobic spoilage. Alternatively, the invention may be used with any susceptible animal feed, whether solid or liquid, e.g. for pigs, poultry or ruminants.

The activity associated with this invention may be found in other strains of L. buchneri, in other species of Lactobacillus, e.g. L. kefir, L. parakefir and L. parabuchneri, L. brevis, L. sake, L. curvatus and possibly also in other genera. This can be established by routine experimentation, on the basis of the information herein.

### Deposits

The Lactobacillus buchneri strain LN1326 was deposited on Nov. 16, 2006 with the Agricultural Research Service (ARS) Culture Collection, housed in the Microbial Genomics and Bioprocessing Research Unit of the National Center for Agricultural Utilization Research (NCAUR), under the Budapest Treaty provisions. The strain was given Patent Deposit No. NRRL B-30989. The address of NCAUR is 1815N. University Street, Peoria, Ill., 61604. The deposit will irrevocably and without restriction or condition be available to the public upon issuance of a patent. However, it should be understood that the availability of a deposit does not constitute a license to practice the subject invention in derogation of patent rights granted by government.

The deposit will be maintained without restriction in the NRRL Depository, which is a public depository, for a period of 30 years, or 5 years after the most recent request, or for the enforceable life of the patent, whichever is longer, and will be replaced if it ever becomes nonviable during that period.

Before describing the embodiments of the present invention in detail, it is to be understood that the embodiments of this invention are not limited to particular compositions or methods of improving digestibility of ensiled forage, which can, of course, vary. It is also to be understood that the terminology used herein is for the purpose of describing particular embodiments only, and is not intended to be limiting.

As used in this specification and the appended claims, the singular forms “a,” “an” and “the” can include plural referents unless the content clearly indicates otherwise. Thus, for example, reference to “a component” can include a combination of two or more components; reference to “feed” can include mixtures of feed, and the like.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Units, prefixes, and symbols may be denoted in their SI accepted form. Numeric ranges recited within the specification are inclusive of the numbers defining the range and include each integer within the defined range.

As used herein, “functional mutant” means a bacterial strain directly or indirectly obtained by genetic modification of, or using, the referenced strain(s) and retaining at least 50% of the activity of a control silage using the referenced strain. The genetic modification can be achieved through any means, such as but not limited to, chemical mutagens, ionizing radiation, transposon-based mutagenesis, or via conjugation, transduction, or transformation using the referenced strains as either the recipient or donor of genetic material.

As used herein, “isolated” means removed from a natural source such as from uninoculated silage or other plant material.

As used herein, “purified” means that a bacterial species or strain is substantially separated from, and enriched relative to: yeasts, molds, and/or other bacterial species or strains found in the source from which it was isolated.

The term “silage” as used herein is intended to include all types of fermented agricultural products such as grass silage, alfalfa silage, wheat silage, legume silage, sunflower silage, barley silage, whole plant corn silage (WPCS), sorghum silage, fermented grains and grass mixtures, etc.

As used herein, “pre-ensiled plant material” means grasses, maize, alfalfa and other legumes, wheat, sorghum, sunflower, barley and mixtures thereof. All of which can be treated successfully with the inoculants of the embodiments of the present invention. The inoculants of the embodiments of the present invention are also useful in treating high moisture corn (HMC).

An embodiment of the invention is a composition for use as a silage inoculant comprising LN1326 or a functional mutant thereof and a suitable carrier. In an embodiment of the invention the composition contains from about 10¹ to about 10¹⁰ viable organisms of the bacterial strain or functional mutant thereof per gram of a pre-ensiled plant material. In a further embodiment of the invention the composition contains from about 10² to about 10⁷ viable organisms of the bacterial strain or functional mutant thereof per gram of a pre-ensiled plant material. In yet a further embodiment the composition contains from about 10³ to about 10⁶ viable organisms of the bacterial strain or functional mutant thereof per gram of a pre-ensiled plant material.

Suitable carriers are either liquid or solid and are well known by those skilled in the art. For example, solid carriers may be made up of calcium carbonate, starch, cellulose and combinations thereof.

An embodiment of the invention is a biologically pure culture of L. buchneri, strain LN1326, having NRRL Patent Deposit No. NRRL B-30989.

Another embodiment of the invention is the combination of LN1326 with other specific bacterial species in the proper ratio to provide both an adequate fermentation of silage or animal feed as well as an enhanced aerobic stability upon exposure of the silage or feed to air. The silage inoculant is an isolated and purified combination of at least one viable strain of the homofermentive lactic acid bacteria Lactobacillus plantarum combined with the heterofermentive bacteria of LN1326. In some embodiments, the silage inoculant will comprise at least 2 to 10 strains of homofermenter and/or heterofermenter. Exemplary strains of L. plantarum include at least one of LP286, LP287, LP329, LP346, LP347, or functional mutants thereof (see, for example, U.S. Pat. No. 6,403,084). Exemplary strains of L. buchneri which could be combined with LN1326 include LN1391, LN4637, LN4750, or functional mutants thereof. The silage inoculant optionally comprises at least one viable strain of Enterococcus faecium, such as, but not limited to, strains EF301, EF202, or functional mutants thereof. The number of viable homofermentive bacteria and heterofermentive bacteria in the inoculant are present in a ratio of from about 1:5 to about 1:15. In some embodiments the ratio is about: 1:6 to 1:14, 1:7 to 1:13, 1:8 to 1:12, 1:9 to 1:11, or 1:10.

Methods of using mixed cultures for improving either fermentation or aerobic stability of silage are disclosed in U.S. Pat. No. 6,403,084, which is herein incorporated by reference.

An embodiment of the invention is a method for improving aerobic stability of silage while also enhancing plant fiber digestion in an animal by feeding an effective amount of a silage that has been inoculated with L. buchneri, strain LN1326, wherein the silage has also been inoculated with a ferulate esterase-containing composition, wherein the ferulate esterase is derived from a ferulate esterase producing bacterial strain or a functional mutant thereof. Methods of using such ferulate esterase producing strains is disclosed in U.S. patent application Ser. No. 11/217,764, herein incorporated by reference. Suitable ferulate esterase producing bacterial strains or functional mutants thereof include, but are not limited to, Lactobacillus strains. Suitable Lactobacillus strains include, but are not limited to, L. buchneri, L. plantarum, L. brevis, L. reuteri, L. alimentarius, L. crispatus, and L. paralimentarius or functional mutants of any of the above strains. Suitable examples of these Lactobacillus strains include, but are not limited to, L. buchneri, strains LN4017 and LN4888; L. plantarum, strains LP678, LP3710, LP3779, and LP7109; L. brevis, strain LB1154, L. reuteri, strain LR4933; L. crispatus, strains L12127, L12350, and L12366, Lactobacillus species unknown, strain UL3050, and mixtures thereof (See U.S. patent application Ser. No. 11/217,764).

The composition that is fed to the animal has been treated with an effective catalytic amount of the ferulate esterase producing bacterial strain or functional mutant thereof as is readily determinable by those skilled in the art in animal husbandry. Animals that are benefited by embodiments of the present invention are mammals and birds, including but not limited to ruminant, equine, bovine, porcine, caprine, ovine and avian species, e.g., poultry.

Embodiments of the present invention are further defined in the following Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

The disclosure of each reference set forth herein is incorporated herein by reference in its entirety.

### EXAMPLES

### Example 1

### Effect of Lactobacillus buchneri strain LN1326 on Aerobic Stability of Greenhouse-Grown Whole Plant Corn Silage

Studies were performed to examine the effectiveness of strain LN1326 to improve the aerobic stability of whole plant corn silage. Strain LN1326 was discovered and identified from a grass sample taken in the United States. Testing conducted on the strain showed that it does not produce ferulate esterase.

Greenhouse grown corn plants (Johnston, Iowa) that had the tassels and ears removed were harvested and transported to the Pioneer Livestock Nutrition Center (PLNC). At the PLNC these plants were chopped and then blended with reconstituted cracked corn at a ratio of 6 parts plant material to 4 parts grain to achieve a mixture of plant material and grain with an approximate dry matter of 35%.

Inoculation: L. buchneri strain LN1326 was grown and either freeze-dried (replicates A&B) or supplied as fresh grown culture (replicate C). LN1326 was solubilized (replicates A&B) and for all 3 studies (A, B & C) adjusted to a standard concentration of 4.54×10⁷ CFU/mL and applied using a 10-cc syringe fitted with a 16-gauge needle at a rate of 2.2 mL/kg of forage. The application rate for all strains was 1×10⁵ CFU/g forage. Propionic acid (88%) was applied at a rate of 4.95 mL/kg fresh forage.

Packets: Approximately 350-400 grams of forage was put into polyethylene packet silos, which were vacuum packed and heat sealed (Dennis et al. (1999) p. 87 In Proc XII Int. Silage Conf. Swedish Univ. of Agric. Sci. Uppsala, Sweden) using a Tilia Food Saver, Professional II model (Tilia Inc. San Francisco, Calif.). The packet silos were stored at room temperature for 30 days until opening.

Aerobic Stability: The method of Honig (Proc. Of the Eurobac. Conf., P. Lingvall and S. Lindgren (ed.) (12-16 Aug. 1986) Swed. Univ. of Agric. Sci. Grass and Forage Report No. 3-1990. Pp. 76-81. Uppsala, Sweden.) was used for measuring aerobic stability. Aerobic stability is defined as the time, in hours, for the silage to heat 1.7° C. after exposure to air. Aerobic dry matter loss (% dry matter) was determined relating the increase in temperature and the time to energy losses occurring in the silage.

### Results

The fermentation patterns observed in these studies are typical of what has previously been described with L. buchneri strains. Generally, the terminal pH values observed after inoculation with L. buchneri are higher than those observed in control. This is likely the contribution of acetic acid produced by L. buchneri versus lactic acid which is the predominant end-product of homofermentative fermentation.

Aerobic stability was improved over the control. An improvement of greater than 24 hours was noted for LN1326.

The degree of heating of the treated silages was considerably less with LN1326. LN1236 reduced the accumulated heat units by more than 50% which resulted in a reduction of more than 40% in the dry matter loss from the silage upon exposure to air.

### SUMMARY

The L. buchneri strain LN1326 used in this study is efficacious in improving aerobic stability of whole plant corn silage. Because of the improved aerobic stability afforded by this strain, substantial improvements in dry matter losses are observed providing an economic advantage to the producer using L. buchneri inoculants.

### Example 2

### Effect of Lactobacillus buchneri strain LN1326 on Aerobic Stability of Grass Silage

Studies were performed to examine the effectiveness of strain LN1326 to improve the aerobic stability of grass silage.

Second cut ryegrass was harvested at PLNC. The grass was determined to have an approximate dry matter of 35%.

Inoculation: Individual strains were supplied as fresh grown overnight culture. Cultures were adjusted to a standard concentration of 5.0×10⁷ CFU/mL and applied using a syringe at a rate of 2.2 mL/kg of forage. The application rate for all strains was 1.1×10⁵ CFU/g forage. Propionic acid (88%) was applied at a rate of 4.4 mL/kg fresh forage.

PVC Silos: PVC silos were filled at a packing density of 0.288 kg DM/silo (100 kg DM/m³); approximately 0.82-0.95 kg/silo. Silos were air infused for 24 hours on days 28 and 42, and opened after 50-60 days of ensiling.

Aerobic Stability: The method of Honig (Proc. Of the Eurobac. Conf., P. Lingvall and S. Lindgren (ed.) (12-16 Aug. 1986) Swed. Univ. of Agric. Sci. Grass and Forage Report No. 3-1990. Pp. 76-81. Uppsala, Sweden.) was used for measuring aerobic stability (see Table 1). ROT is defined as the time, in hours, for the silage to heat 1.7° C. after exposure to air. Cumm_DD is the integral value of the area between the time ROT is attained and the end of the experiment. Aerobic dry matter loss (% dry matter) was determined relating the increase in temperature and the time to energy losses occurring in the silage.

### Results

The fermentation patterns observed in these studies are typical of what has previously been described with L. buchneri strains. Generally, the terminal pH values observed after inoculation with L. buchneri are higher than those observed in control. This is likely the contribution of acetic acid produced by L. buchneri versus lactic acid which is the predominant end-product of homofermentative fermentation.

Aerobic stability was improved over the control. A statistically significant improvement of 119 hours was noted for LN1326, as well as a 96 hour improvement with propionic acid which was included as the positive control (See Table 1).

Grass silages treated with LN1326 and with propionic acid, had significantly less aerobic deterioration than the control grass silages.

### Summary

The L. buchneri strain LN1326 used in this study is efficacious in improving aerobic stability of grass silage. Because of the improved aerobic stability afforded by this strain, substantial improvements in dry matter losses are observed providing an economic advantage to the producer using L. buchneri inoculants.

TABLE 1
Aerobic Stability Parameters of L. buchneri strain LN1326 in Grass Silage. A, B and C
are replicate studies. The average given is calculated across the replicate studies.

| | pH | | | | ROT | | | | Aerobic Dry Matter Loss | | | |
|---|---|---|---|---|---|---|---|---|---|---|---|---|
| | A | B | C | Avg. | A | B | C | Avg. | A | B | C | Avg. |
| Control | 4.23 | 4.27 | 4.24 | 4.24 | 32 | 17 | 44 | 31 | 4.15 | 7.02 | 3.26 | 4.81 |
| Propionic | 4.03 | 4.17 | 4.18 | 4.13 | 137 | 160 | 86 | 127 | 0.29 | 0 | 1.01 | 0.43 |
| Acid | | | | | | | | | | | | |
| LN 1326 | 4.50 | 4.41 | 4.48 | 4.46 | 160 | 129 | 160 | 150 | 0 | 0.99 | 0 | 0.33 |

Underlined items indicate statistical difference from control (P ≦ 0.05)

Having illustrated and described the principles of the embodiments of the present invention, it should be apparent to persons skilled in the art that the embodiments of the invention can be modified in arrangement and detail without departing from such principles. We claim all modifications that are within the spirit and scope of the appended claims.

All publications and published patent documents cited in this specification are incorporated herein by reference to the same extent as if each individual publication or published patent document was specifically and individually indicated to be incorporated by reference.

What is claimed is:

1. A composition for use as a silage inoculant comprising:
a silage quality preserving amount of Lactobacillus buchneri LN1326 or a mutant thereof which retains the silage preservative activity of Lactobacillus buchneri LN1326, and carrier.

2. The composition of claim 1 wherein the composition contains from about 10² to about 10¹² viable organisms per gram wet weight of silage.

3. The composition of claim 1 wherein the composition contains from about 10⁷ to about 10¹⁰ viable organisms per gram wet weight of silage.

4. The composition of claim 1 wherein the composition contains from about 10⁹ to about 10¹⁰ viable organisms per gram wet weight of silage.

5. The composition of claim 1 wherein the carrier is liquid.

6. The composition of claim 1 wherein the carrier is solid.

7. The composition of claim 1 wherein said carrier is a solid carrier selected from the group consisting of calcium carbonate, starch, and cellulose.

8. A biologically pure culture of Lactobacillus buchneri, strain LN1326, having Patent Deposit No. NRRL B-30989.

9. A method for treating silage by inhibiting the growth thereon of spoilage organisms selected from yeasts, molds and spore-forming bacteria, which comprises: adding to said silage a spoilage organism inhibiting amount of the composition of claim 1.

10. A method for treating silage, which comprises adding thereto a microorganism as defined in claim 1.

11. A method according to claim 9, wherein the silage is selected from the group consisting of:
a. grass;
b. maize;
c. alfalfa
d. wheat;
e. legumes;
f. sorghum;
g. sunflower; and
h. barley.

12. A method according to claim 9, wherein said composition is added upon storage of said silage.

13. A method according to claim 9, which comprises storing the treated silage for at least 30 days.

14. A method according to claim 9, wherein the method of ensiling is selected from the group consisting of:
a. ensiling in a bale;
b. ensiling in a bag;
c. ensiling in a bunker;
d. ensiling in a stave silo; and
e. ensiling in a silo.

15. A method according to claim 9, which comprises adding to the silage a silage quality preserving amount of Lactobacillus buchneri strain LN1326, having Patent Deposit No. NRRL B-30989.

16. Silage comprising a silage quality preserving amount of Lactobacillus buchneri LN1326 or a silage quality preserving amount of a mutant thereof.

17. The method of claim 9, wherein said silage is a component of animal feed.

18. A composition for use as a silage inoculant comprising Lactobacillus buchneri LN1326 combined with a ferulate esterase producing bacterial strain or a functional mutant thereof and a suitable carrier.

19. The composition of claim 18, wherein the ferulate esterase producing bacterial strain or functional mutant thereof is a Lactobacillus strain.

20. The composition of claim 19, wherein the Lactobacillus strain or functional mutant thereof is selected from the group consisting of Lactobacillus buchneri, Lactobacillus plantarum, Lactobacillus brevis, Lactobacillus reuteri, Lactobacillus alimentarius, Lactobacillus crispatus, and Lactobacillus paralimentarius.

21. The composition of claim 20, wherein the Lactobacillus buchneri or functional mutant thereof, the Lactobacillus plantarum or functional mutant thereof, the Lactobacillus brevis or functional mutant thereof, the Lactobacillus reuteri or functional mutant thereof, the Lactobacillus alimentarius or functional mutant thereof, the Lactobacillus crispatus or functional mutant thereof, and the Lactobacillus paralimentarius or functional mutant thereof is selected from the group consisting of Lactobacillus buchneri, strain LN4017 (ATCC Patent Deposit No. PTA-6138), Lactobacillus plantarum, strain LP678 (ATCC Patent Deposit No. PTA-6134), Lactobacillus plantarum, strain LP3710 (ATCC Patent Deposit No. PTA-6136), Lactobacillus plantarum, strain LP3779 (ATCC Patent Deposit No. PTA-6137), Lactobacillus plantarum, strain LP7109 (ATCC Patent Deposit No. PTA-6139), Lactobacillus brevis, strain LB1154 (Patent Deposit No. NRRL B-30865), Lactobacillus buchneri, strain LN4888 (Patent Deposit No. NRRL B-30866), Lactobacillus reuteri, strain LR4933 (Patent Deposit No. NRRL B-30867), Lactobacillus crispatus L12127 (Patent Deposit No. NRRL B-30868), Lactobacillus crispatus, strain L12350 (Patent Deposit No. NRRL B-30869), Lactobacillus crispatus, strain L12366 (Patent Deposit No. NRRL B-30870), Lactobacillus species unknown, strain UL3050 (Patent Deposit No. NRRL B-30871), and mixtures thereof.

22. The composition of claim 18, wherein the composition contains from about 10¹ to about 10¹⁰ viable organisms of said bacterial strain or functional mutant thereof per gram of a pre-ensiled plant material.

23. The composition of claim 18, wherein the composition contains from about 10² to about 10⁷ viable organisms of said bacterial strain or functional mutant thereof per gram of a pre-ensiled plant material.

24. The composition of claim 18, wherein the composition contains from about 10³ to about 10⁶ viable organisms of said bacterial strain or functional mutant thereof per gram of a pre-ensiled plant material.

25. A silage inoculant, comprising viable cultures of a homofermentive lactic acid bacteria and a heterofermentive lactic acid bacteria, wherein the homofermentive lactic acid bacteria are isolated and pure Lactobacillus plantarum and the heterofermentive lactic acid bacteria are isolated and pure Lactobacillus buchneri strain LN1326, and wherein the ratio of viable cells of the homofermentive lactic acid bacteria to the heterofermentive lactic acid bacteria ranges from about 1:5 to about 1:15.

26. The silage inoculant of claim 25, wherein the ratio is about 1:8 to about 1:12.

27. The silage inoculant of claim 25, wherein said ratio is about 1:10.

28. The silage inoculant of claim 25, further comprising a viable culture of Enterococcus faecium.

29. The silage inoculant of claim 25, wherein said Lactobacillus plantarum is at least one of: LP286 (ATCC Patent Deposit No. 53187), LP287 (ATCC Patent Deposit No. 55058), LP329 (ATCC Patent Deposit No. 55942), LP346 (ATCC Patent Deposit No. 55943), LP347 (ATCC Patent Deposit No. 55944), or a functional mutant thereof.

30. The silage inoculant of claim 28, wherein said Enterococcus faecium is EF301 (ATCC Patent Deposit No. (55593), EF202 (ATCC Patent Deposit No. 53519), or a functional mutant thereof.

31. The silage inoculant of claim 25, further comprising a carrier suitable for application to silage.

32. The silage inoculant of claim 25, wherein at least two strains of the homofermentive lactic acid bacteria are present in said inoculant.

33. The silage inoculant of claim 32, wherein the at least two strains of homofermentive lactic acid bacteria are at least two of LP286 (ATCC Patent Deposit No. 53187), LP287 (ATCC Patent Deposit No. 55058), LP346 (ATCC Patent Deposit No. 55943), LP347 (ATCC Patent Deposit No. 55944), LP329 (ATCC Patent Deposit No. 55942), or a functional mutant thereof.

34. The silage inoculant of claim 32, further comprising at least one strain of Enterococcus faecium selected from the group consisting of: EF301 (ATCC Patent Deposit No. (55593), EF202 (ATCC Patent Deposit No. 53519), and functional mutants thereof.

35. The silage inoculant of claim 32, wherein at least two strains of homofermentive lactic acid bacteria are present in the inoculant.

36. An animal feed or silage comprising the silage inoculant of claim 25.

37. The animal feed or silage of claim 36 comprising an isolated and purified combination of a viable culture of:
(a) at least two of: LP286 (ATCC Patent Deposit No. 53187), LP287 (ATCC Patent Deposit NQ. 55058), LP346 (ATCC Patent Deposit No. 55943), LP347 (ATCC Patent Deposit No. 55944), LP329 (ATCC Patent Deposit No. 55942), or a functional mutant thereof; and
(b)
(b) Lactobacillus buchneri strain LN1326 or a functional mutant thereof;
wherein the ratio of viable cells of (a) to (b) ranges from about 1:5 to about 1:15.

38. The animal feed or silage of claim 36, wherein the feed is whole plant corn silage or high moisture corn.

39. A method of treating animal feed or silage, comprising administering the silage inoculant of claim 25 to the feed or silage at about 1×10⁴ to 1×10⁵ CFU/g of feed or silage.

40. The method of claim 39, wherein the feed or silage is whole plant corn silage or high moisture corn.

41. A method of improving animal performance, comprising feeding the animal the animal feed of claim 36.
